(19) United States
(12) Patent Application Publication
(10) Pub. No.: US 2024/0157039 A1
(43) Pub. Date: May 16, 2024

(54) METHODS, APPARATUSES, AND SYSTEMS FOR ASPIRATING AIRWAYS

(71) Applicant: Board of Regents, The University of Texas System, Ausitn, TX (US)

(72) Inventors: R. Lyle Hood, San Antonio, TX (US); Robert De Lorenzo, San Antonio, TX (US); Tyler Bull, San Antonio, TX (US); Saketh Ram Peri, San Antonio, TX (US); Alejandro Medina-Montemayor, San Antonio, TX (US); Zach Fallon, San Antonio, TX (US)

(21) Appl. No.: 18/504,509

(22) Filed: Nov. 8, 2023

Related U.S. Application Data
(60) Provisional application No. 63/424,063, filed on Nov. 9, 2022.

(51) Int. Cl. A61M 1/00

(52) U.S. Cl. CPC A61M 1/741; A61M 1/60; A61M 1/784; A61M 2205/7536

(57) ABSTRACT

The present disclosure includes methods, apparatuses, and systems for aspirating the airway of a patient. The apparatus includes a main body having a pump and a storage cannister housing coupled to the main body and containing a storage container that is at least partially collapsible. In some configurations, the main body can include a pressure sensor, a controller in communication with the pressure sensor, and having a processor, a memory, and a power source in communication with the controller. The storage cannister housing can include a first end coupled to the main body, and a second end having a weighted portion and configured to be coupled to the first end to permit free rotation, such that the second end gravitationally rotates.

### RELATED APPLICATIONS

This application claims priority to U.S. Provisional Patent Application No. 63/424,063 filed Nov. 9, 2022. The entire contents of the above-referenced disclosure is incorporated herein by reference in its entirety.

### TECHNICAL FIELD

The present disclosure relates generally to methods, apparatuses, and systems that assist in the clearing of a patient's airway. More specifically, the present disclosure relates to methods, apparatuses, and systems that assist in aspirating the airway of a patient and where the apparatus is orientation independent and/or has an improved flow path.

### BACKGROUND

Airway injuries, which may result from blunt force and penetrating injuries to the neck and chest, can be life threatening conditions, particularly on a battlefield. The presence of concomitant severe injuries and non-specific symptoms and signs for this type of injury may delay diagnosis and lead to early fatal outcome due to asphyxiation from airway obstruction or death from tension pneumothorax, or late sequela such as airway stenosis and recurrent pulmonary infections.

Airway obstruction and compromise is the second leading cause of preventable battlefield death, which can be attributed to the unavailability of sufficiently powerful portable suction systems, among other factors. Therefore, prompt diagnosis is mandatory for the survival of these patients. However, treatment of these patients is challenging and includes securing a patient airway that will allow adequate ventilation and then repairing the injury with a smaller impact on the respiratory function and the quality of life of the patient.

Portable suction devices available in the market are either manually powered and less efficient, or battery powered and bulky/heavy for combat medics to carry in their kits. Examples of portable suction devices include the Laerdal Compact Suction Unit® 4 (LCSU® 4) by Laerdal Medical; the SSCOR Quickdraw® by SSCOR, Inc.; and the Clario Airway Suction Pump by Medela AG.

The LCSU® 4 includes a collection cannister supported by a wire bracket. The collection cannister can come in a 300 mL size or an 800 mL size container when extra suction volume is needed. The LCSU® 4 weighs between 3.3 to 4.3 pounds depending on the collection cannister size and provides between 50 mmHg to 550 mmHg vacuum pressure for about 45 minutes when powered by batteries. The collection cannister must also be correctly oriented to fit into the wire bracket. An air flow rate of 30 L/min can be obtained with the device. The overall dimensions of the LCSU® 4 are 18.5 cm×26.2 cm×8.12 cm with a 300 mL cannister and 23.6 cm×19 cm×23.6 cm with an 800 mL cannister.

The SSCOR Quickdraw® Tactical requires 10 AAA sized batteries to power the device for 60 to 100 minutes. A maximum negative pressure of >500 mmHg and a low negative pressure setting of 80 mmHg to 100 mmHg can be achieved with the device. The drainable collection cannister can hold a maximum of 300 mL. The collection cannister must also be correctly oriented. The SSCOR Quickdraw® Tactical weighs 2.6 pounds and has overall dimensions of 27 cm×11 cm×11 cm.

The Clario Airway Suction Pump by Medela AG is intended for home health use and can operate for about 50 minutes when powered using the rechargeable battery. A low vacuum setting of 135 mmHg, a medium vacuum setting of 270 mmHg, and a maximum vacuum setting of 600 mmHg can be achieved with the device. The collection cannister can hold a maximum of 550 mL and must also be correctly oriented during operation. A fluid flow rate of 15 L/min can be obtained with the device. The Clario Airway Suction Pump weighs about 2.0 kilograms and has overall dimensions of 22.3 cm×25.5 cm×9.5 cm. Market assessments and surveys conducted by the inventors have demonstrated that combat medics require a lightweight, compact, efficient, and reliable portable suction alternative for the battlefield because combat medics are required to carry everything on their backs. Similarly, civilian EMS providers need a lightweight, reliable, and effective means of clearing debris such as saliva and vomitus from the airway of critical patients. Thus, portability, effectiveness, and ruggedness are key attributes for an airway suction device.

Current designs fall short on many aspects because they were developed using poor/outdated technology. The designs are typically heavy, bulky, provide inadequate suction, and have a short battery life. This results in unnecessary patient suffering and can lead to death from suffocation due to inadequate aspiration of debris or the inability to gain control of the patient's airway in a timely manner.

In view of the foregoing, it is apparent that there exists a need for a device and method to clear airways of patients that overcomes, mitigates, or solves the above problems in the art. Embodiments described herein fulfill this and other needs in the art, which will become apparent to the skilled artisan once given the following disclosure.

### SUMMARY

This disclosure includes implementations of methods and configurations of apparatuses and systems for aspirating the airway of a patient that address the deficiencies of the devices described above. Non-limiting examples of conditions that benefit from this disclosure include, but are not limited to, tracheobronchial injuries, asphyxiation from airway obstruction, and tension pneumothorax. Certain embodiments are directed to an apparatus for aspirating an airway of a patient, the apparatus comprising a pump portion operatively coupled to a cannister portion. The device being configured to be orientation independent, which is defined by the ability to remain operable when the orientation of the device is changed, that is the device is designed to remain operable when rotated in the x, y, and/or z plane. The pump portion can include one or more pressure sensor, controller, pump, and/or memory. The controller can be in communication with the pressure sensor. The controller can include a processor and a memory. A power source, e.g., a battery, can be in communication with the controller and optionally included in the pump or cannister portion. The cannister portion can include or house a liquid collection cannister (“cannister” or “low-pressure area”). The cannister portion or the liquid collection cannister can have a first or proximal end coupled to the pump portion. In other embodiments the cannister can be coupled to the pump portion or pump assembly at both ends. In certain aspects there may be two pump portions or pump assemblies at both ends of the cannister.

Certain embodiments are directed to a liquid collection cannister comprising a first end and a second end connected by a body wall forming a cannister lumen for receiving fluid during use, the body wall having one or more inlets configured to be coupled with one or more aspiration tubes, at least one of the first end or second end having at least two hydrophobic filters coupled to one or more vacuum inlet, the hydrophobic filters being configured to retain liquid in the cannister and allow gas to be removed from the cannister during use. Further embodiments are directed to a liquid collection cannister comprising a first end and a second end connected by a body wall forming a cannister for receiving and retaining fluid during use, the body wall having one or more inlets configured to be coupled with one or more aspiration tubes. The first end having a hydrophobic filter assembly comprising at least one hydrophobic filter and the second end having a hydrophobic filter assembly having at least one hydrophobic filter, the hydrophobic filters are configured to retain liquid in the cannister and allow gas to be removed from the cannister during use. In certain aspects the cannister is a cylinder. The cannister can have a length of 3 cm to 100 cm and a radius of 0.5 cm to 50 cm. In certain aspects the first end and second end can have at least two hydrophobic filters each. The at least two hydrophobic filters can be positioned on opposite sides of the interior face of the first end and/or second end. The hydrophobic filters can be configured as vacuum conduits connecting the vacuum source to the cannister to provide vacuum to an aspiration tube. The hydrophobic filters can be paper filters. In other aspects the hydrophobic filters comprise a paper filter on a mesh support. The hydrophobic filters can be two-dimensional disks or three-dimensional hollow filters, the three-dimensional hollow filters forming vacuum conduits. The three-dimensional hollow filters can project from the first end and the second end into the cannister and are operably coupled to the vacuum source forming vacuum conduits. The cannister can further comprise an aspiration coupling for attaching an aspiration tube or the like. The cannister can further comprise an aspiration tube coupled to the cannister via the aspiration coupling. The aspiration tube can have a suction tip. In certain aspects, filter(s) can be position at one or both ends of the cannister to provide suction during changes in orientation.

The controller can be configured to measure a pressure at the inlet and shut off the pump when the measured pressure falls below a threshold pressure. The apparatus of any embodiment described herein can further include a light source coupled to or integrated into the handle, the pump portion, cannister portion or any combination thereof. In certain aspects, the apparatus of any embodiment described herein can further include an outer cover configured to be coupled to the pump portion or cannister portion and enclose that portion. The interior walls of the liquid collection cannister can be modified or coated, for example the interior wall of the cannister can be coated, completely or partially with super absorbent polymers, pH reagents, and/or oxygen-sensitive luminescent materials that will assist with demobilizing fluid contents located inside for evacuation from device and assist with content characterization. Certain embodiments can include design elements that provide for characterization of fluid content, these elements include but are not limited to (i) a separate sample chamber couple to the cannister, either upstream or downstream with respect to the cannister; (ii) sensors that measure metrics that help identify fluid sources, such as pH or blood oxygenation, this allows for determination if accumulated fluids include arterial blood (indicating a dangerous hemorrhage), vomit, or other components that may dictate clinical response; (iii) a multi-lumen tubing can be utilized to provide a conduit for wiring, etc. and/or (iv) a “periscope-like” orifice in the housing located over the cannister for assisted viewing of cannister content.

In certain aspects, multi lumen tubing can be included as a suction catheter equipped with additional features without becoming large and bulky, the features include, but are not limited to (i) fiber optic cables inserted through a small channel of the tubing—eliminating the need for an LED, switch, and a battery to be embedded into the catheter; and/or (ii) electrical controls and/or connections for the operation of the suction tip, this may also allow for small data lines to be passed through the small channel to transmit information to a CPU within the pump housing.

A centrifugal pump or diaphragm pump can be configured to generate a vacuum pressure of at least or about 400, 500, 600, to 700 mmHg, including all values and ranges there between. The controller can be configured to evacuate water, vomitus, solid pieces, solid particulates, and/or blood at a flow rate of at least or about 0.5, 0.75, 1.0, 1.25, 1.5, 2.0, 3.0, 4.0, 5.0 to 10 L/min, including all values and ranges there between. The liquid collection cannister can have a storage volume of at least 0.2, 0.5, 1.0, 1.5, 2.0 L to 3.0 L. The processor can be configured to determine patient condition via optical and/or electrochemical analysis. The pump portion, liquid collection cannister, and/or outer cover includes a surface coating with one or more characteristics including one or more of an anti-reflective, a camouflage, an electromagnetic shielding, or combinations thereof. The apparatus of any embodiment described herein can further include a muffler system coupled to the main body to reduce operating noise. The controller can be configured to provide a pressure range for treating pneumothorax (e.g., 5 to 20 mmHg).

Certain embodiments are directed to methods for aspirating an airway of a patient, the method comprising: (a) providing any one of the apparatuses described herein; (b) inserting the suction tube into the airway of the patient; (c) aspirating a fluid from the airway using any one of the apparatuses described herein. The method can further include (e) detecting a patient condition via optical and/or electrochemical analysis using any one of the apparatuses described herein.

The term “coupled” is defined as connected, although not necessarily directly, and not necessarily mechanically; two items that are “coupled” may be unitary with each other.

The terms “a” and “an” are defined as one or more unless this disclosure explicitly requires otherwise.

The term “substantially” is defined as largely but not necessarily wholly what is specified (and includes what is specified, e.g., substantially 90 degrees includes 90 degrees and substantially parallel includes parallel), as understood by a person of ordinary skill in the art. In any configuration or implementation of the present devices, apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus, device, or kit that “comprises,” “has,” “includes” or “contains” one or more elements possesses those one or more elements but is not limited to possessing only those elements. Likewise, a method that “comprises,” “has,” “includes” or “contains” one or more steps possesses those one or more steps but is not limited to possessing only those one or more steps.

Further, an apparatus, device, or structure that is configured in a certain way is configured in at least that way, but it can also be configured in other ways than those specifically described.

Any configuration or implementation of any of the present devices, apparatuses, kits, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the configurations described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the configurations depicted in the figures.

FIG. 1. An illustration of one example of an aspiration device.

FIG. 2. An illustration of a non-limiting example of a hydrophobic filter that can be used in the current invention.

FIG. 3. An illustration of one example of a liquid collection cannister.

FIG. 4. An exploded illustration of one example of an aspiration device incorporating a liquid cannister of FIG. 3.

FIG. 5. An illustration of an aspiration device assembly illustrating one example of an aspiration device.

FIG. 6. An illustration of a side view of one example of a double walled cannister.

FIG. 7. An illustration of a perspective view of one example of a double walled cannister.

FIG. 8. An illustration of a top view of one example of a double walled cannister

FIG. 9. An illustration of an example of a lock and pop cannister design.

FIG. 10. An illustration of an example of the working of a lock and pop cannister design.

FIG. 11. An illustration of an example of a lock and pop cannister disposal process.

FIG. 12. An illustration of the height of hydrophobic poles of a lock and pop cannister design.

FIG. 13. An illustration of the types of hydrophobic filter assemblies.

FIG. 14. An illustration of the use of types of hydrophobic filter assemblies.

FIG. 15. An illustration of one example of a two pump apparatus design.

### DESCRIPTION

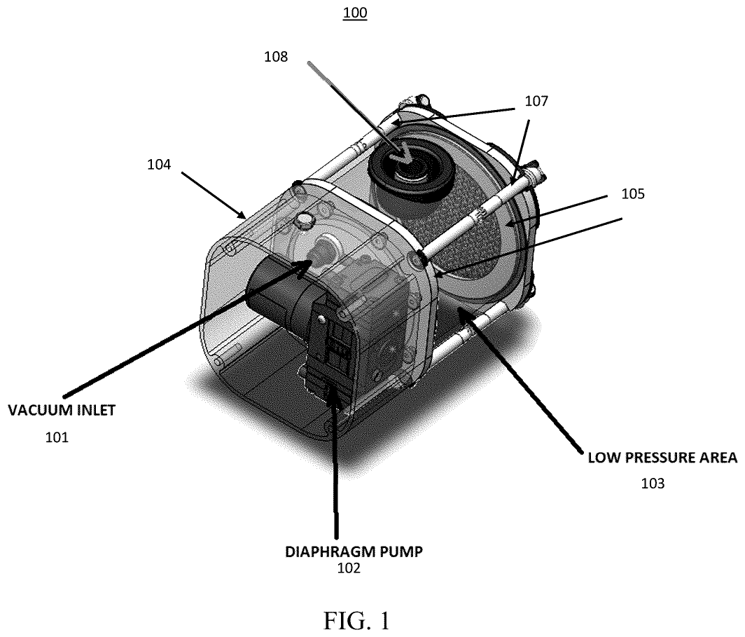

FIG. 1 illustrates one example of an aspirating device 100 comprising a liquid collection cannister having dual hydrophobic filters at each end. The device of FIG. 1 has a pump or control portion/assembly 104 operatively connected to a cannister or storage portion. The pump portion including vacuum inlet or port 101 coupled to pump 102 and further coupled to the cannister or storage portion including a low-pressure area 103, e.g., a liquid collection cannister. The low-pressure area/cannister 103 can have hydrophobic filter assembly 105 at each end of the cannister. Hydrophobic filter assemblies 105 are configured to allow liquid to enter the cannister but not exit the cannister while allowing gases to move freely through the device and enter and exit the cannister. Low-pressure area/cannister 103 has at least one aspiration connection that is configured to couple an aspiration tube to the low-pressure area/cannister 103 to effect aspiration when in use. The low-pressure area/cannister 103 can be removable. The low-pressure area/cannister can be held in place by cannister supports 107. The cannister supports can include compression hinges configured to allow the low-pressure area/cannister 103 to be connected, secured, and removed/replaced when appropriate.

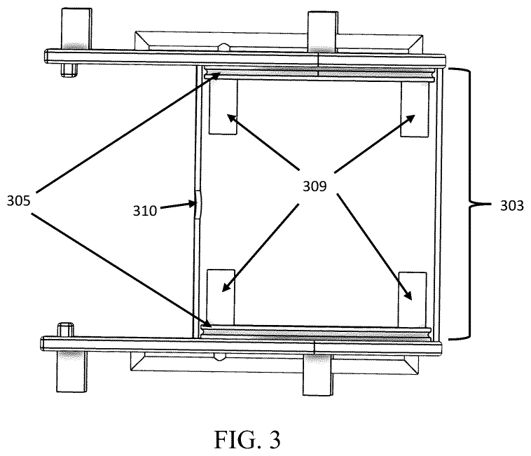

FIG. 3 illustrates one example of a cannister design. Cannister 303 has walls forming a cannister lumen. Cannister 303 has two hydrophobic filter assemblies 305 at each end. In this embodiment hydrophobic filter assemblies 305 include two or more vacuum conduits 309. Vacuum conduit 309 is liquid impermeable/gas permeable and function as a hydrophobic filter allowing gas to pass and retaining liquid in the cannister. Vacuum conduits 309 can be positioned so that at least one conduit is not submerged in the collected liquid which imparts an orientation independence. Each hydrophobic filter assembly 305 can have 1, 2, 3, 4, 5, 6, 7, 8, 9, or more vacuum conduits 309 that can be positioned in various regular or irregular patterns across the inner face or along the inner perimeter of the hydrophobic filter assembly 305. The cannister 303 can also include an aspiration inlet 310 that can be operatively connected to an aspiration tube.

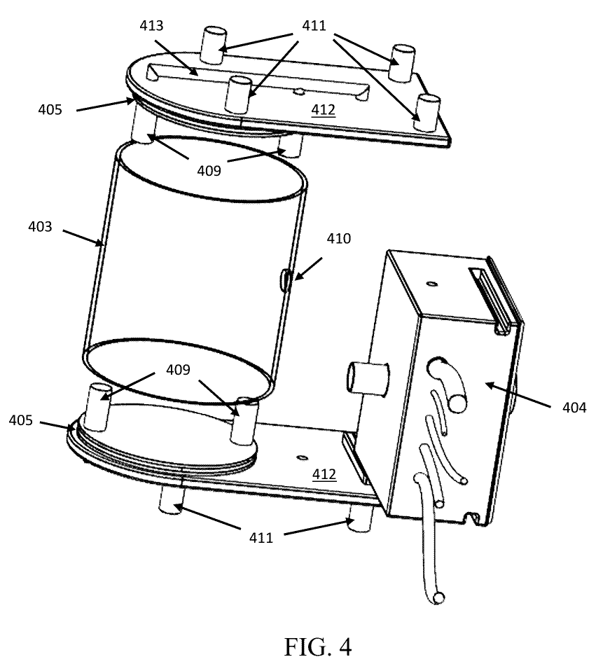

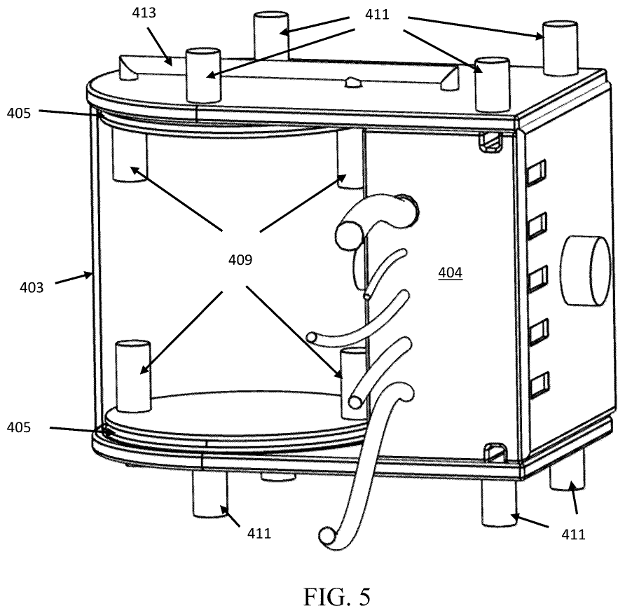

FIG. 4 and FIG. 5 illustrate one example of an aspiration device employing a cannister as described in FIG. 3. FIG. 4 is an exploded illustration and FIG. 5 is an assembled illustration. The illustrated device has pump assembly 404 that is connected to cannister assembly 403/405 by a cannister support 412. Cannister support 412 has legs or supports 411 and a vacuum path 413. Vacuum path 413 connects vacuum conduits 409 to a vacuum source in the pump assembly 404. Cannister 403 has hydrophobic filter assemblies 405 at both ends. Hydrophobic filter assembly 405 has one or more vacuum conduits 409 which in certain embodiment comprise a hydrophobic filter and provide vacuum to the cannister. Cannister 403 has an aspiration connection that can be connected to an aspiration tube.

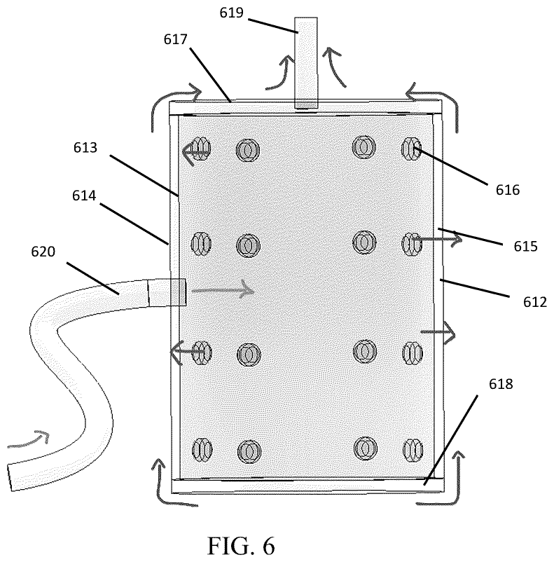

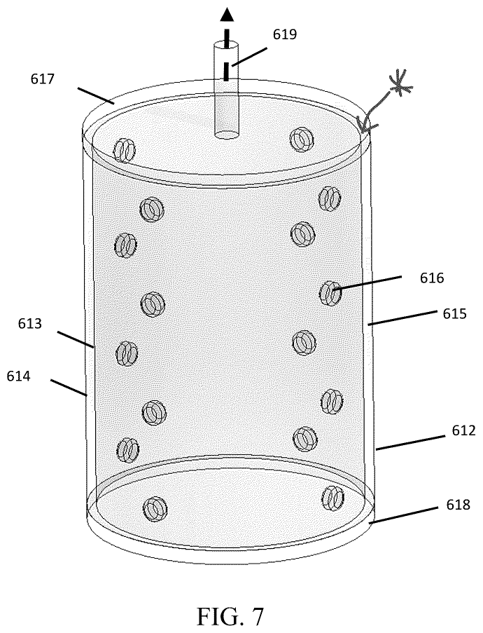

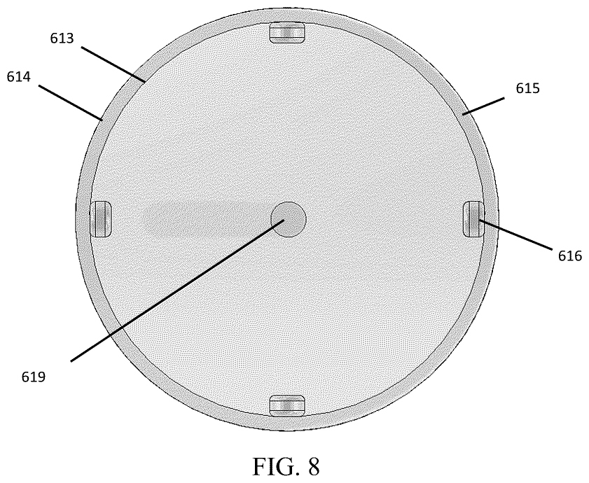

FIG. 6 to FIG. 8 illustrate a double hull cannister design. The double hull wall cannister 612, having an inner wall 613 and an outer wall 614 forming a wall space 615, the wall space 615 providing a channel for air transfer within the wall of the cannister 612. A plurality of gas ports 616, including one-way check valves or mini hydrophobic assemblies, are positioned across the inner wall 613 of the double hull cannister 612 to allow for air transfer from the cannister lumen to wall space 615 and inhibiting liquid flow into a wall space 615 formed by the inner cannister wall 613 and outer cannister wall 614. In certain aspects, gas ports 616 are regularly or irregularly disperse along the circumference and/length of the inner cannister wall. There can be as few as 2 and as many as 100 gas ports. In certain aspects the top lid 617 and/or bottom lid 618 have cavity(ies) fluidly connected with wall space 615 which continue the air transfer from the aspiration tube 620 to cannister 612 to the wall space 615 to the top lid 617 to the exit tube 619 which exits the cannister 612 and goes to the pump or other vacuum source.

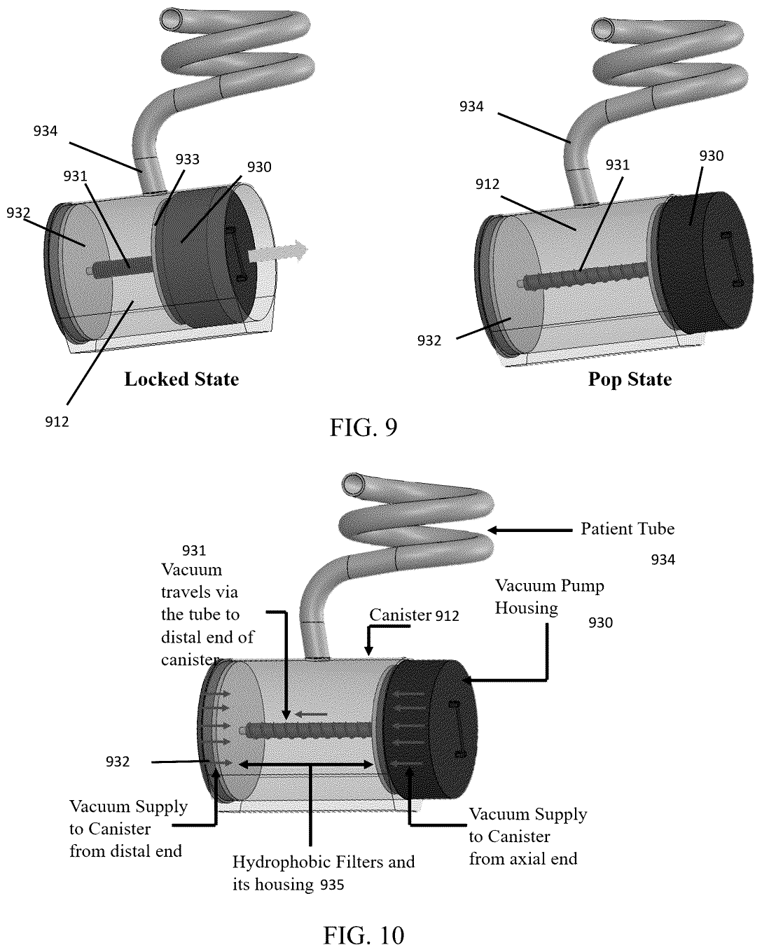

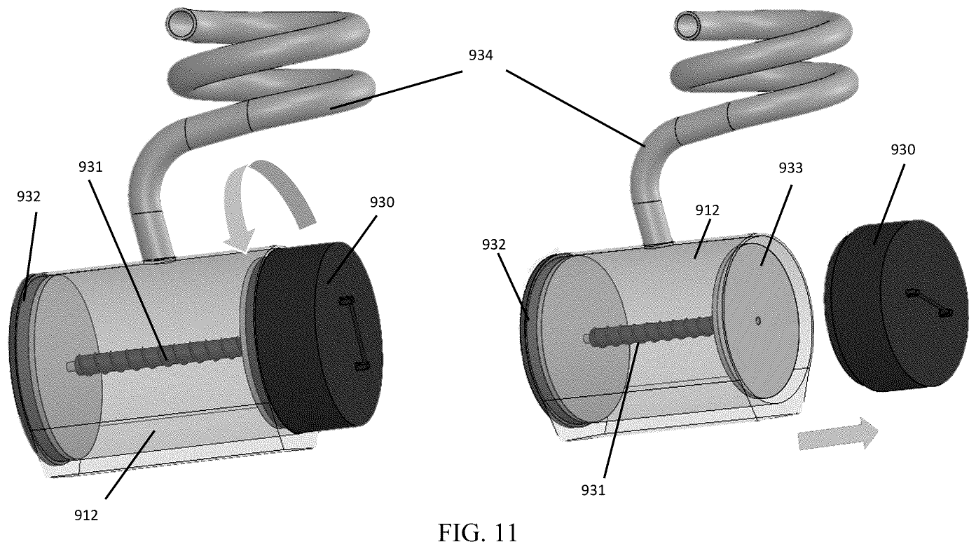

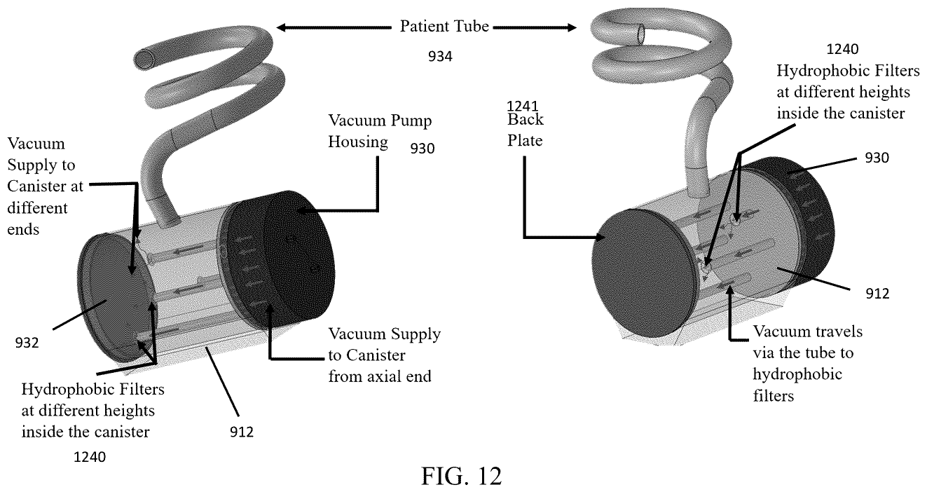

Certain embodiments are directed to a lock and pop design, see FIG. 9, FIG. 10, FIG. 11 and FIG. 12. The lock and pop design has a moveable vacuum pump housing 930 that translates along the long axis of the cannister 912. Vacuum pump housing 930 is coupled to an extendable cannister vacuum tube 931. In a locked configuration the vacuum pump housing 930 is stored in cannister 912 and the cannister vacuum tube 931 is in a contracted position. In a Pop configuration the vacuum housing 930 extended to end of the cannister 912 and is secured to the cannister 912 with the vacuum tube 931 in an extended position. Cannister vacuum tube 931 extends from the vacuum pump housing 930 to the distal end of cannister 912. Cannister 912 can have a bottom lid 932, optionally coupled to a filter 935 and a top lid 933. Bottom lid 932 having a cavity to transfer the vacuum force to the bottom of the cannister. Top lid 933 is operably connected to pump housing 930. Bottom lid 932 can be connected to a back plate 1241. In certain aspects filters 935 are positioned at the bottom, top, or bottom and top of the cannister. In certain aspects filter(s) 935 are a syringe filter, a disc membrane filter, or another form of filter configured to allow gas and vacuum to be transmitted but retain liquid in the cannister. Cannister 912 is operably coupled to an aspiration or patient tube 934. A vacuum is generated within the cannister from one or both ends (distal, proximal, or distal and proximal) which supplies a vacuum on the aspiration or patient tube 934.

Referring to FIG. 12, tubular filter components 1240 of different heights can be operably coupled to the top lid, bottom lid, or top lid and bottom lid. There can be 1, 2, 3, 4, 5, 6, 7, 8, 9, 10 or more tubular filter components.

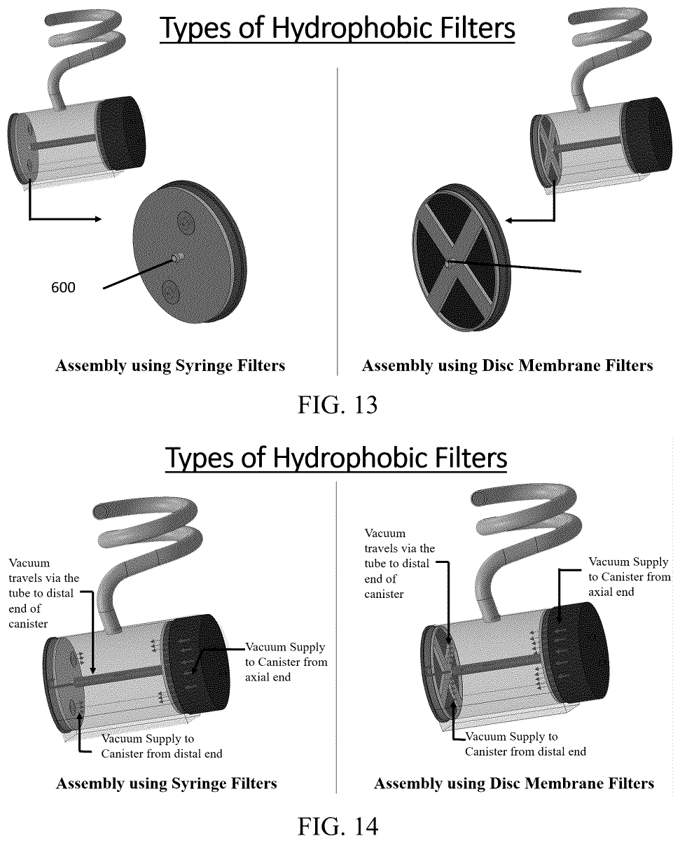

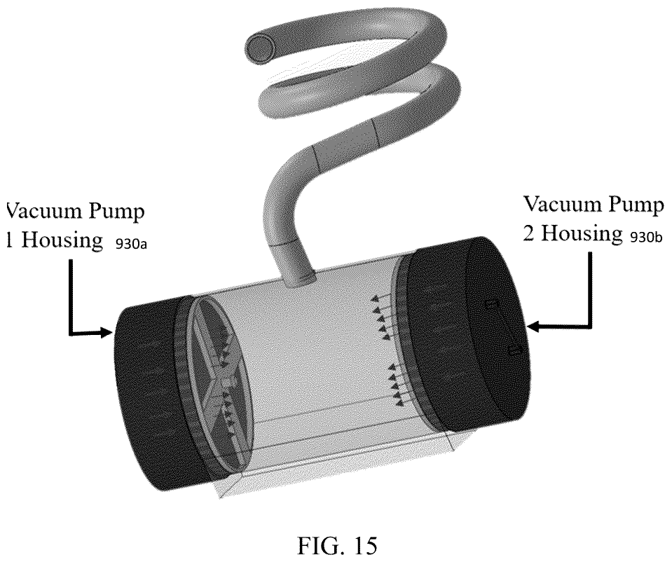

FIG. 13 illustrates two examples of filters that can be used alone or in combination. A filter can include a syringe filter or a disc membrane filter. The filters can have a vacuum port configured to allow vacuum produced or introduced at one end to be applied to the distal end. In certain aspects a vacuum tube is operably connected to pump housing and distal lid component. FIG. 14 illustrates the vacuum applied to the filters.

FIG. 15 illustrates an example of a two pump housing configuration having a first 930a and second 930b pump housing. Each pump housing can individually be configured in a pop and lock configuration or be stably mounted at the ends of a cannister.

In some configurations, a controller is configured to measure a pressure at the inlet and shut off the pump when the measured pressure falls below a threshold pressure. In some configurations, the controller is configured to evacuate water, vomitus, solid pieces, solid particulates, and/or blood at a flow rate of at least 0.5 L/min. In some configurations, the controller is configured to provide a pressure range for treating pneumothorax. In some configurations, the processor is configured to determine a patient condition via optical and/or electrochemical analysis.

In some configurations, the aspiration device outer surface includes a surface coating selected from the group of surface coating characteristics consisting of anti-reflective, camouflage, electromagnetic shielding, and combinations thereof. In some configurations, a muffler system can be operatively coupled to the aspiration device to reduce operating noise.

In some configurations, the aspiration device has an overall dimension of 5, 10, 20, 30, 40, 50, 60, 70, 80, 90 to 100 cm×2, 5, 10, 15, 20, 25, 30, 35, 40, 45, to 50 cm×2, 5, 10, 15, 20, 25, 30, 35, 40, 45, to 50 cm or less. In some configurations, the aspiration device has an overall weight of at least, at most, or about 0.25, 0.5, 1, 2, 3, 4, 5, 6, 7, 8, 9, to 10 kg, including all values and ranges there between. In certain aspects the aspiration device has an overall weight of 0.25 to 2 kg. In other aspects the aspiration device has an overall weight of 2 to 7.5 kg.

In some implementations, a method for aspirating an airway of a patient includes (a) providing an aspiration device described herein; (b) inserting the suction tube into the airway of the patient; (c) aspirating a fluid from the airway using an aspiration device described herein. In some implementations, the method further includes detecting a condition in a patient via optical and/or electrochemical analysis of a fluid or liquid collected or during collection using an aspiration device described herein.

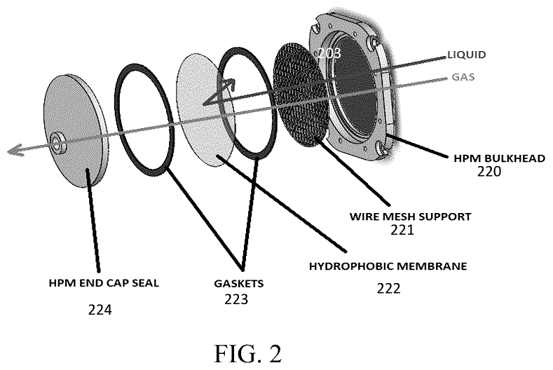

Hydrophobic Filters. FIG. 2 illustrates one example of a hydrophobic filter. Membranes are hydrophobic to the extent that, firstly, no water penetrates into the filter membrane structure and, secondly, no closed water film can form on the filter membrane surface and thereby restrict or stop the air flow through the filter membrane. If water vapor is carried out of the solution with the air flow, it condenses at least in part on the filter membrane and all housing and pipeline parts and must be discharged. At high air flows in particular, cells, cell debris or particulate substances, for example nutrients or residues thereof, are carried along onto the filter membrane surface and block the pore structure of the filter membrane. The filtering media may, in one embodiment, include a sheet-like, porous fabric made for example in the shape of a membrane or any other porous structure, made from a single layer or from a stack of multiple layers of the same material or different materials. The hydrophobic structure may be fabricated from any suited material known in the art. Hydrophobic filter fabrics may for example be made from a PTFE material, wherein however also other materials are conceivable. The hydrophobic filter material can be composed of a composite material, such as, for example, Gortex™, Teflon™, or nylon. PVDF (polyvinylidene fluoride), PVC, PTFE, ePTFE (expanded polytetrafluoroethylene), PP or PE, cellulose ester.

Hydrophobicity in the context of this text is to be understood as the tendency of the filtering media to adsorb little or no water. Whereas a hydrophilic filtering media exhibits an affinity for water and readily adsorbs water, a hydrophobic filtering material has the opposite response to water interaction compared to hydrophilic materials. Hydrophobic materials have little or no tendency to adsorb water and water tends to bead on their surfaces (i.e. to form discrete droplets). Hydrophobic materials generally possess low surface tension values and lack active groups in their surface chemistry for formation of “hydrogen-bonds” with water. Water or other aqueous solutions hence generally may not pass the hydrophobic structure of the filtering media, such that water or other aqueous solutions and also debris particles or the like are blocked by the filter such that air or another gaseous flow is filtered. the filter material 144 can be made of a hydrophobic or hydrophilic material or can be coated with a hydrophobic or hydrophilic material for selective filtering.

The above specification and examples provide a complete description of the structure and use of exemplary configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the present devices, apparatuses, kits, and methods are not intended to be limited to the particular forms disclosed. Rather, they include all modifications and alternatives falling within the scope of the claims, and configurations other than the one shown may include some or all of the features of the depicted configuration. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one configuration or may relate to several configurations.

The claims are not intended to include, and should not be interpreted to include, means-plus- or step-plus-function limitations, unless such a limitation is explicitly recited in a given claim using the phrase(s) “means for” or “step for,” respectively.

### REFERENCES

The following references, to the extent that they provide exemplary procedural or other details supplementary to those set forth herein, are specifically incorporated herein by reference.
1. C. Prokakis, et al. “Airway trauma: A review on epidemiology, mechanisms of injury, diagnosis and treatment” Journal of Cardiothoracic Surgery, 9(1):117, 2014.
2. Eastridge, B. J., et al, “Death on the battlefield (2001-2011): Implications for the future of combat casualty care” J Trauma & Acute Care Surg, 73(6), S431-S437, 2012.
3. Peake J B. “Beyond the Purple Heart: continuity of care for the wounded in Iraq” N Engl J Med, 352(3):219-222, 2005.
4. Champion H R, et al, “A profile of combat injury”. J Trauma, 54(5): S13-S19, 2003.
5. A. T. Simpson. “Transporting lazarus: Physicians, the state, and the creation of the modern paramedic and ambulance 1955-73.” J History of Medicine & Allied Sciences, 2013.
6. Calkins, M. D. “Evaluation of possible battlefield suction pumps for the far-forward setting.” Military medicine, 167(10): 803, 2002.

1. A liquid collection cannister comprising:
a first end and a second end connected by a body wall forming a cannister for receiving fluid during use,
the body wall having one or more inlet configured to be coupled with one or more aspiration tubing,
at least one of the first end or second end having at least two hydrophobic filters coupled to a vacuum inlet, the hydrophobic filters configured to retain liquid in the cannister and allow gas to be removed from the cannister during use.

2. The cannister of claim 1, wherein the first end and second end has at least one hydrophobic filters coupled to a vacuum inlet.

3. The cannister of claim 1, wherein the cannister is a cylinder.

4. The cannister of claim 3, wherein the cylinder has a length of 10 cm to 100 cm and a radius of 2 cm to 50 cm.

5. The cannister of claim 1, wherein the first end comprises at least two hydrophobic filters.

6. The cannister of claim 5, wherein the at least two hydrophobic filters are positioned on opposite sides of the first end.

7. The cannister of claim 1, wherein the second end comprises at least two hydrophobic filters.

8. The cannister of claim 7, wherein the at least two hydrophobic filters are positioned on opposite sides of the second end.

9. The cannister of claim 1, wherein the hydrophobic filters are paper filters.

10. The cannister of claim 1, wherein the hydrophobic filters comprise a paper filter on a mesh support.

11. The cannister of claim 1, wherein the hydrophobic filters are two-dimensional disks or three-dimensional hollow filter.

12. The cannister of claim 1, wherein the hydrophobic filters are three-dimensional hollow filters that project from the first end and the second end into the cannister and are operably coupled to the vacuum source.

13. The cannister of claim 1, further comprising an aspiration tube coupled to the inlet.

14. The cannister of claim 13, further comprising a suction tip coupled to the aspiration tube.

15. An apparatus for aspirating an airway of a patient, the apparatus comprising the liquid collection cannister of claim 1 operably coupled to a pump portion.

16. The apparatus of claim 15, wherein the pump portion comprises a controller portion, a vacuum source, and an optional power source configured to provide a vacuum to the liquid collection cannister during use.

17. The apparatus of claim 16, wherein the vacuum source is a diaphragm pump.

18. The apparatus of claim 16, wherein the vacuum source is configured to generate a vacuum pressure of at least 400 to 550 mmHg.

19. The apparatus of claim 15, where the liquid collection cannister has a storage volume of at least 0.2 L to 10 L.

20. The apparatus of claim 15, further comprising a suction tip attachment operably coupled to the inlet of the liquid collection cannister.

21. The apparatus of claim 20, wherein the suction tip includes a filter for trapping debris.

22. The apparatus of claim 15, wherein the apparatus has an overall dimension of 3 cm to 100 cm×5 cm to 50 cm×5 cm to 50 cm.

23. The apparatus of claim 15, wherein the apparatus has an overall weight of 0.25 kg to 10 kg.
